(19) United States
(12) Patent Application Publication
(10) Pub. No.: US 2007/0259035 A1
(43) Pub. Date: Nov. 8, 2007

(54) Colored liquid-filled soft capsules and method of manufacture thereof

(75) Inventor: Timothy B. Chiprich, Charlotte, NC (US)

Correspondence Address: HOFFMANN & BARON, LLP, 6900 JERICHO TURNPIKE, SYOSSET, NY, 11791, US

(73) Assignee: Leiner Health Products, LLC.

(21) Appl. No.: 11/827,129

(22) Filed: Jul. 9, 2007

Related U.S. Application Data
(62) Division of application No. 10/754,458, filed on Jan. 8, 2004.

(51) Int. Cl. A61K 9/48; A61P 43/00

(52) U.S. Cl. 424/456

(57) ABSTRACT

The present invention relates to a coated liquid-filled soft capsule containing a colored shell and method for making same. The shell material contains a colorant incorporated in the material in an amount sufficient to provide a visual contrast between the capsule shell and any liquid fill that escapes from the capsule shell and resides on an exterior surface of the capsule shell. This allows leaking capsules as well as other capsules that get contaminated from the leaking capsules to be identified before they are coated. This prevents coating problems in pan and/or continuous coaters associated with leaking and/or contaminated capsules. The present invention also produces pharmaceutically elegant capsules uniform in appearance without the necessity of coloring the coating.

### BACKGROUND OF THE INVENTION

The present invention relates to soft capsules and, more specifically, to colored liquid-filled soft capsules and methods for their manufacture.

Capsule dosage forms include soft capsules and hard capsules. Capsules are widely used in the pharmaceutical industry as an oral dosage form for the administration of many different types of active pharmaceuticals, vitamin products, and nutritional supplements. The capsules are often filled with an active ingredient in the form of a liquid, a powder, or a powder suspended in liquid. Hard capsules are often made of unplasticized or low-plasticized gelatin and water to form a stiff capsule that is typically filled with either powder or liquid. Soft capsules are often made of highly plasticized soft elastic gelatin and often contain a liquid or semisolid ingredient. These capsules are often referred to as “softgel” capsules.

Soft capsules are now a commonly used dosage form. Soft capsules are a unique dosage form that can provide distinct advantages over more traditional dosage forms such as tablets, hard-shell capsules, and liquids. These advantages include patient compliance and consumer preference, improved bioavailability, speed of product development in many cases, shortened manufacturing time, enhanced drug stability due to less exposure of the active ingredient to oxygen, excellent dose uniformity, and product differentiation, for example through novel shapes.

There are, however, some disadvantages to soft capsules. These disadvantages include the need for specialized manufacturing equipment and the resulting higher cost of manufacturing.

There are additional difficulties encountered with soft capsules when they are liquid filled. Very often, a manufacture of soft capsules will encounter the problem of “leakers.” Leakers, as the name implies, are liquid filled soft capsules that develop some sort of breach or hole in the capsule barrier causing the liquid filler material to reach the outside of the capsule. The liquid on the outside of the capsule interferes with the adhesion of any additional coating to the capsule, such as an enteric coating or finishing coating. In addition, the leakers similarly contaminate other capsules that they come in contact with during the manufacturing process.

The soft capsule shell can be breached by physical handling during the manufacturing process. However, most leakers are due to imperfect seal formation. Leaking capsules cannot be successfully coated. For example, soft capsules are often coated with an enteric coating to improve the aesthetic appearance of the capsule, prevent odor and aftertaste of the liquid or semisolid ingredient, and may also have a non-enteric finishing layer to prevent the capsule from sticking to other adjacent capsules inside the packaging.

However, when coatings are applied to soft gelatin capsules, the capsules tend to come out hazy looking with some areas of the coatings appearing whiter than other parts. As a result, the capsules look non-uniform in appearance, with the natural amber color of the gelatin showing through the hazy non-uniform looking coating.

To make the products pharmaceutically elegant, the enteric coating and/or finishing coating is often pigmented in a way that the final coated soft gelatin capsules are opaque and uniformly colored. Unfortunately, adding pigments to the enteric coating and/or finishing coating causes several problems. The problems include excessive coating times, coating splits from swollen capsules and brittleness of the coatings.

For example, the pigmented enteric coating and/or finishing coating has to be applied until the coated soft capsules appear uniform. The amount necessary to attain a uniform appearance is often much higher than the amount needed to make a capsule having the desired enteric properties. This causes the coating time to be excessive.

Another problem encountered is related to the excessive coating times. The gelatin material used in the soft capsule is hygroscopic and most coating is done using water as a processing aid. During the application of coating, the soft capsule will often absorb moisture from the additional aqueous coating solution. This absorption of moisture can cause the capsule to swell. The swelling is most evident at the capsules' seams because they are thinner than the rest of the capsule shell. The swelling can then cause the coating to split at the area of the capsules seam.

Accordingly, there is a need for an improved soft capsule and method for their manufacture.

### SUMMARY OF THE INVENTION

In accordance with the present invention, a coated liquid-filled soft capsule is provided. The coated capsule includes a liquid fill, a soft capsule shell encapsulating the liquid fill and a coating applied on the exterior surface of the capsule shell. The capsule shell is formed from a material which further contains a colorant incorporated in the material. The colorant is in an amount sufficient to provide a visual contrast between the capsule shell and any liquid fill that escapes from the capsule shell and resides on an exterior surface of the capsule shell.

The liquid fill can be in the form of a liquid or a suspension of a solid in a liquid.

In another aspect of the invention, an enteric coated liquid-filled soft capsule is provided. The enteric coated capsule includes a liquid fill, a soft capsule shell encapsulating the liquid fill and an enteric coating applied on the exterior surface of the capsule shell. The capsule shell contains gelatin and a colorant in the gelatin in an amount sufficient to provide a visual contrast between the capsule shell and any liquid fill that escapes from the capsule shell and resides on an exterior surface of the capsule shell.

In a further aspect of the invention, a method for making a coated liquid-filled capsule is provided. The method includes encapsulating a liquid fill with a soft capsule shell. The capsule shell is formed from a material which further contains a colorant incorporated in the material. The colorant is in an amount sufficient to provide a visual contrast between the capsule shell and any liquid fill that escapes from the capsule shell and resides on an exterior surface of the capsule shell. The next step is to determine that the liquid fill has not escaped the encapsulation of the capsule shell such that the capsule is suitable for a coating applied on said exterior surface. A coating is applied to the capsule on the exterior surface.

In yet another aspect of the invention, a method for making an enteric coated liquid-filled soft capsule is provided. The method includes encapsulating a liquid fill with a soft capsule shell. The capsule shell contains gelatin and a colorant incorporated in the gelatin in an amount sufficient to provide a visual contrast between the capsule shell and any liquid fill that escapes from the capsule shell and resides on an exterior surface of the capsule shell. The next step is to determine that the liquid fill has not escaped the encapsulation of the capsule shell such that the capsule is suitable for a coating applied on the exterior surface. An enteric coating is applied to the capsule on the exterior surface.

The coated capsules of the invention and their method of making provide distinct improvements over the prior art. By coloring the soft capsule shell a different color than the internal liquid filling, it is easier to detect and remove leakers that would compromise the quality of a batch of coated capsules.

In addition, by coloring the capsule shell itself, as opposed to adding a pigment layer after the formation of the capsule shell, there is no need to coat the capsule with a thick colored layer. Instead, only a very thin coating layer that imparts the desired enteric and/or protective properties to the capsules can be applied to provide a pharmaceutically elegant capsule. The application of a thinner aqueous enteric and/or protective coat provides less moisture available for the capsule shell to absorb. The absorption of less moisture causes less swelling of the capsule shell and, therefore, less likelihood that the coating will develop seam splits.

### DETAILED DESCRIPTION OF THE INVENTION

The invention relates to a coated liquid-filled soft capsule. The coated capsule includes a liquid fill encapsulated with a soft capsule shell. The exterior surface of the soft capsule shell is coated with one or more layers of coating.

The liquid fill can be any liquid suitable for a soft capsule. The liquid fill is typically an active ingredient in the form of a liquid or a solid suspended in a liquid. The liquid for suspending the solid can be any liquid suitable for suspending the active ingredient. Examples of suitable liquids include water miscible and water immiscible liquids. Examples of water miscible encapsulatable liquids include polyethylene glycols and polysorbate 80. Examples of water immiscible encapsulatable liquids include vegetable and animal oils. Examples of active ingredients include active pharmaceutical agents, vitamins, minerals, antioxidants, enzymes, immunostimulants, weight loss products, energy products or other nutritional supplements.

The pharmaceutical agent can be any drug for treating or preventing any disease, condition or disorder. Examples of pharmaceutical agents include analgesics, anti-allergic agents, antivirals, anti-fungals, immunosuppressants, anticoagulants, antidepressants, antineoplastic agents, cardiovascular agents, antibiotics and anti-inflammatory agents.

A vitamin is any organic substance which is typically essential for the normal growth and activity of humans. Vitamins are typically either fat-soluble or water-soluble. The vitamin can be any vitamin. Examples of vitamins include, but are not limited to, vitamin A (retinol), B1 (thiamine), B2 (riboflavin), B complex, B6 (pyridoxine), B12 (cobalamin), C (ascorbic acid), D (cholecalciferol), E (tocopherol), F (linoleic acid), G, H (biotin), and K, and choline, folic acid, inositol, niacin, pantothenic acid, and para-aminobenzoic acid.

Minerals are naturally occurring inorganic substances which are typically essential to the nutrition of humans. The mineral can be any mineral. Examples of minerals include, but are not limited to, boron, calcium, chromium, copper, iron, magnesium, manganese, molybdenum, nickel, phosphorus, selenium, silicon, tin, vanadium, and zinc. Substances containing minerals, for example limestone, can be used as the source of the mineral.

Antioxidants are substances which inhibit oxidation. Typically, antioxidants inhibit oxidation by quenching free radicals. Examples of antioxidants include, but are not limited to, Vitamin E, Vitamin C, beta carotene, Coenzyme Q10, tocopherols, ascorbyl palmitate, Alpha Lipoic Acid, Bilberry Extract, Ginkgo Biloba Extract and Rosemary Extract.

Enzymes are proteins which functional as biochemical catalysts. Enzymes can, for example, aid in digestion, assist in building bone and other tissues, catalyze the production of energy or exchange of oxygen, and carbon dioxide. Enzymes generally function as oxidoreductases, transferases, hydrolases, lyases, isomerases or ligases. Examples of enzymes include, but are not limited to, lipase, lactase, protease, cellulase, maltase, sucrase and amylase.

Immunostimulants are substances for enhancing the immune system against a disease, illness or condition. Examples of diseases, illnesses or conditions in which immunostimulants can be used include, for example, a bacteria or viral infection, and cancer. Examples of immunostimulants include, but are not limited to, Echinacea, β-glucan, Vitamin C, Zinc, Goldenseal, Arabinogalactan and Astragalus.

Weight loss products are substances which assist in weight loss. The weight loss product can, for example, prevent the digestion of certain foods, such as fats, finction as an appetite suppressant, or increase metabolism rate. Examples of weight loss products include, but are not limited to, Conjugated Linoleic Acid, Chromium, 5-Hydroxytryptophan, Chitosan, Garcinia and Citrus aurantium.

Energy products are substances for boosting energy and/or for renewing and conserving energy reserves. These products are especially useful for those suffering from, for example, fatigue and/or lethargy. Examples of substances for boosting energy include, but are not limited to, Guarana, Mate, Cola Nut, Ginseng, Rhodiola, Schisandra, Cordyceps, and B Vitamins.

A nutritional supplement is generally any substance taken to improve the health of the individual. Examples of such substances include nutritional oils. The nutritional oil can be obtained from an animal, plant or vegetable. Example of such oils include fish oil, flaxseed oil, borage oil, evening primrose oil, perilla oil, any oil suitable for human consumption containing essential fatty acids, and combinations thereof. Examples of essential acids include linoleic acid, alpha linolenic acid, gamma linolenic acid, conjugated linoleic acid and arachidonic acid. The nutritional oils can be used in combination with a stabilizer. The stabilizer can be any substance which prevents degradation of the nutritional oil. An example of a stabilizer is an antioxidant.

In a preferred embodiment, the nutritional oil is obtained from fish. An example of nutritional oil from fish is omega-3-fatty acids. Examples of omega-3-fatty acids include eicosapentaenoic acid and docosahexaenoic acid. The omega-3-fatty acids can be used either alone or in combination with any of the antioxidants described above, including for example, Coenzyme Q10, tocopherols, ascorbyl pa imitate, and rosemary extract.

In another embodiment, the nutritional oil is obtained from vegetables. A example of a nutritional oil from vegetables is omega-6-fatty acids. An example of omega-6-fatty acids is linoleic acid. In a preferred embodiment, the omega-3-fatty acid is used in combination with an omega-6-fatty acid. In yet another preferred embodiment, the nutritional oil contains omega-3 fatty acids, omega-6 fatty acids, essential fatty acids, or combinations thereof

The liquid fill can also include any pharmaceutically acceptable excipient. Examples of suitable excipients include, but are not limited to the following: triglyceride oils of animal, vegetable or mineral origin, beeswax, paraffin wax, stearates, polyethylene glycols, non ionic surface active agents, sterols, or ethers.

The liquid fill is encapsulated in a soft capsule shell. The soft capsule shell is formed from a material suitable for encapsulating the liquid fill. The material is present in the capsule shell in an amount to yield a soft capsule shell sufficient to encapsulate and protect the liquid fill.

The material is present in the capsule shell in an amount of at least about 20% by weight, more preferably at least about 25%, and most preferably at least about 30% by weight of the shell. The material is present in the capsule shell in an amount of at most about 85% by weight, preferably at most about 70%, more preferably at most about 65%, and most preferably at most about 60% by weight of the capsule shell.

Suitable materials for encapsulating the liquid fill include heat sealable polymers and gelatin. Examples of heat sealable polymers include, but are not limited to, modified starches, cellulosic polymers and carrageenans.

Preferably, the material is gelatin. The gelatin can be natural gelatin, chemically modified gelatin, enzymatically modified gelatin, or combinations thereof.

Natural gelatin is a proteinaceous biopolymer which is derived from denatured collagen. The natural gelatin can be from any source. Typically, the natural gelatin is obtained from an animal source. For example, gelatin can be obtained from horns, hooves, hides, skin, bones, membranes or connective tissue of an animal. The animal can be any animal. Examples of suitable animals include, but are not limited to cows, horses, roosters, fish and pigs. Gelatin can also be obtained from human recombinant collagen.

Chemically modified gelatin include gelatin which has been modified by the addition of one or more chemical groups. The chemical group can be any group known to those skilled in the art for chemically modifying gelatin. For example, appropriate chemical groups include, but are not limited to, succinate groups, acyl groups, phthalate groups, carbamoylate groups or combinations thereof.

Enzymatically modified gelatin include gelatin which has been modified by enzymes or where the collagen used to make gelatin has been enzymatically modified. The enzyme can be any enzyme known to those skilled in the art for enzymatically modifying gelatin. For example, appropriate enzymes groups include, but are not limited to, proteases such as trypsin and pepsin or combinations thereof.

The material which forms the capsule shell typically further includes water. Water is present in the original material mass before the capsules are made, in an amount sufficient to allow the processing of the material on the encapsulation machine. After the capsules are formed the majority of the moisture is removed during the drying process.

To yield a capsule shell having the softness and flexibility in accordance with the present invention, water is typically present in the shell at a minimum amount of about 5% by weight, preferably a minimum of about 6%, and more preferably a minimum of about 7% by weight. The maximum amount of water present in the shell is about 15% by weight, preferably about 12% by weight, and more preferably about 10% by weight.

The water typically has a plasticizing effect on the material. However, water is generally too volatile to rely on as the sole plasticizer. Therefore, a non-volatile plasticizer or blend of plasticizers can be added to the material which forms the capsule shell.

The non-volatile plasticizer can be any plasticizer compatible with the material of the capsule shell. For example, the non-volatile plasticizer can be glycerine, maltitol, sorbitan, sorbitol or similar low molecular weight polyhydric alcohols, and mixtures thereof. The ratio of plasticizer to material typically determines how hard or soft the shell will be.

The ratio of plasticizer to material in the shell is in an amount sufficient such that the capsules are not too hard, such that the capsules are brittle and crack if stressed during shipping and handling, and are not too soft, such that the capsules become deformed during shipping and handling. Generally, the non-volatile plasticizer is preferably present in the capsule shell from about 8% to 65% by total weight of the capsule shell, most preferably from about 10% to 35% by total weight of the capsule shell.

The material which forms the capsule shell further contains a colorant. The colorant is added to the material forming the capsule shell such that a visual contrast is provided between the capsule shell and any liquid fill that escapes the shell and resides on the exterior surface of the capsule shell. The colorant can include, for example, a pigment or a dye, or a combination of a pigment and a dye.

Pigments are generally not water-soluble or less water soluble than dyes. Dyes are generally more water-soluble than pigments. The use of a pigment in the shell material typically makes the shell opaque as well as color the shell, whereas dyes usually just color the shell.

Examples of colorants useful in the present invention include titanium dioxide, zinc oxide, iron oxides, iron hydroxides, calcium carbonate, calcium sulfate, curcumin, riboflavin, tartrazine, quinoline yellow, carmoisine, indigo carmine, chlorophylls, copper complexes of chlorophylls, lissamine green, caramel, charcoal, carotenoids, xanthophylls, anthocyanins, alumina, aluminum powder, annatto extract, bismuth oxychloride, bronze powder, canthaxanthin, chromium-cobalt-aluminum oxide, chromium hydroxide green, cochineal extract, carmine, copper powder, ferric ammonium citrate, ferric ammonium ferrocyanide, ferric ferrocyanide, guanine, logwood extract, mica, potassium sodium copper chlorophyllin, pyrogallol, ptrophyllite, talc, annatto extract, FD&C dyes, aluminum lake forms of FD&C dyes, D&C dyes, and aluminum lake forms of D&C dyes.

The amount of colorant present in the shell material is any amount sufficient to visually color the capsule shell. Typically, the amount of colorant present in the shell is from about 0.1% to about 2.0%.

In a preferred embodiment, the colorant is titanium dioxide. Preferably the titanium dioxide is present in the capsule shell material in an amount at least from about 0.1%, preferably at least about 0.3%, and more preferably at least about 0.5%. The capsule shell material contains at most about 2.0% titanium dioxide, preferably at most about 1.5%, and more preferably at most about 1.0% titanium dioxide.

The colorant used for the capsule shell material typically depends on the nature of the liquid fill. To provide a visual contrast, the color of the capsule shell is a different color than the liquid fill. Thus, the colorant is incorporated in the capsule shell material in an amount sufficient to provide a visual contrast between the capsule shell and any liquid fill that escapes from the capsule shell and residues on an exterior surface of the capsule shell.

For example, if the liquid fill is brown in color, the shell can be a white opaque color. Thus, if any brown liquid fill escapes from the capsule shell and resides on an exterior surface of the capsule shell, it will be readily apparent whether a capsule contains a breach in the integrity of the shell. For example, fish oil turns from a practically clear oil to a dark brown oil as it oxidizes after being exposed to air. The brown oxidized fish oil is easy to detect on a white shell, whereas it is very hard to detect on an unpigmented shell, because gelatin is naturally amber in color and thus, there is no visual contrast between the oxidized fish oil and the amber shell.

In addition to providing a visual contrast between the shell and any liquid fill that escapes the shell, the shell can be colored such that the shell assists in preventing degradation of the active ingredient of the liquid fill. For example, the omega-3 fatty acids in fish oil are susceptible to degradation upon exposure to light. Thus, coloring of the shell can minimize exposure of the active ingredients to light.

The liquid fill can further contain a colorant. The colored liquid fill can, for example, provide an additional visual contrast. The colorant useful for the liquid fill can be any of the colorants described above or any other pharmaceutically acceptable colorant.

The material which forms the capsule shell can further contain extenders and/or plasticizers. The plasticizer can be any of those plasticizer described herein.

The extender can be any extender which is compatible with the material. Examples of extenders include natural or modified natural biopolymers and synthetic polymers. Natural biopolymers include, for instance, cellulose, starch, starch derivatives, bacterial polysaccharides such as xanthan gum and gellan gum and vegetable gums such as guar gum, locust bean gum, gum tragacanth and gum Arabic and animal derived polymers such as chondroitin sulfate, hyaluronic acid, heparin, collagen and chitosan. An example of a modified natural biopolymer is modified cellulose. Examples of synthetic polymers include carbon chain polymers of the vinyl and acrylic types as well as heterochains of the polyoxide and polyamine types.

The material which forms the capsule shell can also include any pharmaceutically acceptable excipient. The excipient can be any excipient compatible with the material. The excipient useful with the material of the capsule shell include those excipients described above, as well as other excipients such as flavorings and essential oils, as described herein, to impart desirable odors to the capsules.

A coating is applied on the exterior surface of the soft capsule shell. The coating can contain one or more layers. Any coating suitable for a soft capsule can be applied to the capsule. The coating can provide, for example, waterproofing and sealing, smoothing, polishing, enteric protection and/or delayed release properties to the liquid-filled capsule. The delayed release can be affected by, for example, temperature or pH. In a preferred embodiment, the coating is an enteric coating.

The coating can be made by any standard coating ingredient known to those skilled in the art. Coating ingredients include, but are not limited to, fats, fatty acids, waxes, shellac, ammoniated shellac, cellulose acetate phyhalates, celluosics, vinyls, glycols, acrylics and carbohydrate polymers, polymers and co-polymers containing methacrylic acid and methacrylic acid alkyl esters, hydroxypropylmethyl cellulose (HPMC) and combinations thereof.

In a preferred embodiment, the coating substantially lacks a colorant. In this specification, “substantially lacks a colorant” means that the coating contains no colorant or contains very little colorant such that any colorant present does not impart a color to the coating. Coloring of the capsule shell provides pharmaceutically elegant capsules uniform in appearance without having to color the coating. Coloring the capsule shell allows the application of a thinner coating over the shell which provides less moisture for the capsule shell to absorb. The absorption of less moisture causes less swelling of the capsule shell and, therefore, less likelihood that the coating will develop seam splits.

The coated capsule can further contain a finishing coating. Typically, the finishing layer is applied to the coated-capsule to make the capsule pharmaceutically elegant. Examples of substance suitable for use in a finishing coating include, but are not limited to cellulosics, vinyls, glycols, acrylics and carbohydrate polymers and/or combinations thereof.

The coating and/or finishing coating can further contain plasticizers, such as polyhydric alcohols, acetate esters, phthalate esters, glycerides and oils, and/or processing aids, or any of the plasticizers described herein. Examples of processing aids include glidants/anti-tack agents such as talc and glyceryl monostearate, as well as surfactants/wetting agents and delivery vehicles such as water or organic solvents.

The coating and/or finishing coating can further contain flavors and/or fragrances to impart an appealing taste and/or odor to the coated liquid-filled capsule. For example, if the active ingredient is fish oil in a gelatin soft capsule, the odor of fish oil and certain components of gelatin may be considered to be unpleasant to some individuals. Thus, flavors and fragrances can be used to mask the unpleasant odor and taste of the active ingredient and capsule shell material.

The flavor can be a natural or artificial flavor and typically also is pleasantly fragrant. An example of an edible pleasantly fragrant flavor is vanilla. Other edible fragrant flavors include, but are not limited to, essential oils such as peppermint or rosemary oils.

In a preferred embodiment, the capsule contains fish oil in a gelatin shell. The shell contains about 30% to about 70% gelatin, about 6% to about 10% water, about 10% to about 35% polyhydric alcohol plasticizer and about 0.1% to about 1.5% titanium dioxide.

In another aspect of the invention, the invention provides a method for making a coated liquid-filled soft capsule of the present invention. The capsule shell is formed from a material which further contains a colorant incorporated in the material. The colorant can be incorporated in the shell material by any method. Typically, a colorant, such as titanium dioxide, is added to the molten shell material (e.g., gelatin) before the encapsulation process. The colorant can be incorporated, for example, by mixing the colorant with the material. The colorant is added in an amount sufficient to provide a visual contrast as described above.

The first step in the method includes encapsulating a liquid fill with a soft capsule shell. The liquid fill can be encapsulated with a soft capsule shell by any method known in the art. For example, a soft capsule can be made using a standard rotary die soft gelatin capsule machine as described in the book The Theory and Practice of Industrial Pharmacy, ed. Lachman, et al., 2nd Ed., Pt. II, 404-420, Lea & Febiger, 1976. Additional methods include using an old plate process (see The Theory and Practice of Industrial Pharmacy, ed. Lachman, et al., 2nd Ed., Pt. II, 405, Lea & Febiger, 1976), as well as Globex type seamless capsule machines, which makes large microcapsules (see U.S. Pat. No. 5,254,294), non-standard rotary die machines, which uses extrusion technology to make gel ribbons (see U.S. Pat. Nos. 6,183,845 and 6,340,473), and other methods for making capsules which use high frequency, ultrasonic, or induction welding to seal the capsules (see U.S. Pat. No. 6,352,719). The above-listed U.S. Patents and book are hereby incorporated by reference.

Periodic checks are usually performed during manufacturing of a soft capsule to insure that the capsules contain a sufficient wet seal thickness, as the capsules are formed, filled and sealed along the capsules seams. The checks for seal thickness can be performed by any method known to those in the art. For example, checks for seal thickness can be performed by measuring a cross section of the capsules with a microscope fitted with a measuring gauge.

Generally, a sufficient seal thickness is at least about 0.006 inches, preferably at least about 0.01 inches, and more preferably at least about 0.015 inches. The maximum seal thickness has no upper limit in terms of functionality, but generally is equal to or less than the thickness of the non-seal portion (i.e., sides) of the capsule shell. Typically, the wet shell thickness of the capsule is from about 0.025 inches to about 0.035 inches.

The next step in the method is to determine that the liquid fill has not escaped the encapsulation of the capsule shell. The determination can be performed by manual or mechanical means, such as, for example, visually or with an instrument that capable of detecting the contrast. For instance, since there is a visual contrast between the capsule shell and liquid fill, it is apparent whether any liquid fill escapes from the capsule shell.

For example, if the liquid fill is fish oil and the capsule shell contains titanium dioxide, the capsule shell can be opaque white in color. Fish oil is considered to be a semi-drying oil which turns from a practically clear oil to a dark brown sticky oil as it oxidizes after being exposed to air. Thus, any fish oil that escapes the capsule shell and onto an exterior surface of the capsule shell will oxidize and form a sticky brown film on the exterior surface of the capsule shell. Due to the visual contrast between the color of the capsule shell and the liquid fill, capsules which contain a breach in the integrity of the shell, as well as any other capsules which became contaminated from contact with leaking capsules, can be identified and discarded. The remaining capsules (capsules which do not contain a breach in the shell integrity and which are not contaminated from contact with the liquid fill from a leaking capsule) are suitable for applying a coating on the exterior surface of the capsule shell.

The coating can be applied to the exterior surface of a capsule shell by any method known to those in the art. For example, the capsule can be coated with standard coating machinery. Examples of standard coating machinery includes perforated pan coaters and fluid bed coaters as described in the book The Theory and Practice of Industrial Pharmacy, ed. Lachman, et al., 2nd Ed., Pt. II, 377-382, Lea & Febiger, 1976 as well as continuous coaters such as the one described in the Thomas Engineering Continuous Coater Specification sheet (available at http://www.thomaseng.com/ctc.htm). The enteric coating and, if present, the finishing or protective coating can be applied by any method known to those in the art. An example of a suitable coating method is described in the book Practical Course in Film Coating of Pharmaceutical Dosage Forms with Eudragit, Lehmann et al., 2nd Ed., Section 1.3.9 Enteric coating, 64-66, Rohm, 1996. The above references books are hereby incorporated by reference.

### EXAMPLE

The following non-limiting example has been carried out to illustrate preferred embodiments of the invention. The example includes the preparation of colored soft gelatin capsules filled with fish oil and having a coating.

TABLE I

% w./w.

| Ingredient | Preferred | Most Preferred |
|---|---|---|
| Gelatin, NF | 20-85 | 30-70 |
| Glycerin | 8-65 | 10-35 |
| Purified Water | 5-15 | 6-10 |
| Titanium Dioxide | 0.1-2.0 | 0.1-1.5 |

The above listing of the ingredients, and preferred, and most preferred ranges of amounts of the ingredients are utilized in the finished, dried shell of the capsules of the present invention. The amount of water should be sufficient to produce a flowable gelatin mass while in a molten state at a viscosity of about 5,000 to about 25,000 cps at approximately 140.degree. F.

In preparing the capsules of the present invention, the amount of water shown in Table I for the finished product is modified, since during drying, water is lost so that the final parameters of formulation are as shown in Table I.

The gelatin formula of the present invention is prepared using conventional techniques. The gelatin, glycerin, titanium dioxide and water are placed into a suitable mixer, such as a pony mixer or jacketed mixer-melter vessel with mixing and vacuum capabilities, and are mixed until the liquids are absorbed into the solids and are considered “crumbed” (meaning fluffy swollen pieces). The crumbed gelatin formula components are then melted under vacuum at approximately 29.5″ HG at 140-200° F. until molten which may take from 1 to 5 hours, depending upon the quantity of the materials.) Alternatively, the gelatin, glycerin and titanium dioxide can be added while mixing to preheated water in the same type of melter to speed up the process. The gel mass is then transferred to a holding tank and maintained at approximately 140° F. Alternatively, the gelatin mass is made the same way as above with the exception that the titanium dioxide is added to the uncolored gel mass in the holding tank with the aid of a suitable high shear mixer.

The mass of gelatin is fed into a standard rotary die process soft gelatin encapsulation device. The gelatin is passed through heated tubes into spreader boxes which spread the gel onto cold rotating drums forming a gelatin ribbon. The thickness of the gel ribbon is maintained a target thickness between 0.025 inches and 0.035 inches. The gel ribbon is then fed through lubricated guided rollers which feed the ribbon between an injection wedge and die rolls.

The fill material, which is not less than 1000 mg Fish Oil containing approximately 12% Docosahexaenoic Acid and approximately 18% Eicosapentaenoic Acid in this example (Note—alternatively capsules with higher levels of Eicosapentaenoic Acid and Docosahexaenoic Acid as well as capsules containing just one or the other would also work with this invention), is injected through the heated wedge while the ribbon goes through the die rolls.

The heat of the wedge along with the pressure of the dies cause capsules to be simultaneously cut, filled and sealed. Machine parameters are adjusted as appropriate to maintain an appropriate wet seal thickness that will result in a robust capsule.

The wet seal thickness is typically at least 0.006 inches thick as measured by examining a cross section of the capsule shells under a microscope fitted with a measurement gauge. The capsules are then transferred by conveyor belt into drying tumblers (equipped with heated and/or humidity controlled air) to begin the drying process. The capsules are transferred automatically through the drying tumbler and, if necessary (i.e. if not left in the tumblers to dry to completion) are spread on trays. The trayed capsules are then placed in a controlled humidity area (approximately <15% Relative Humidity) to finish drying. The capsules lose water as they come to equilibrium with the humidity of the drying area.

The capsules are considered to be sufficiently dry when they are firm enough to resist deformation when packed into a shipping carton, but not so dry that they are capable of cracking during shipping and handling. Typically such capsules will be in the hardness range of approximately 7 to 10 Newtons if measured with a Durometer. The capsules are examined, and if there is any evidence of excessive amounts of lubricant and/or fish oil on their surface they can be washed with appropriate organic solvents and/or be polished in a tumbler with oil absorbing cloths. At this point in time, the capsules are ready to be packed in bulk cartons to await the coating process.

The capsules that are to be coated undergo an additional inspection process. Each carton of capsules is examined before it is emptied into the coating machine. If any evidence of leaking capsules is found, as indicated by the easily detectable presence of brown oxidized fish oil residue on the surface of the white capsules, the capsules are rejected before applying a coating layer.

In an appropriate container 385.000 gm of Methacrylic Acid-Ethyl Acrylate Copolymer (1:1) Dispersion 30% is mixed with 272.030 gm of purified water and is mixed until a uniform suspension is achieved. Add 57.750 gm of Glycerol Monostearate Emulsion (PLAS II from Emerson Resources, Inc. which contains a proprietary mixture of Water, Glycerol Monostearate, Triethyl Citrate, Polysorbate 80, Methyl Parabens and Propyl Parabens) and 27.720 gm of Triacetin and stir for not less than sixty minutes.

Three and one half (3.5) kg. of capsules are placed into a 19″ O'Hara Lab Coater or other similar tumbling device. While tumbling at approximately 13 R.P.M. the capsules are allowed to tumble with warm air passing over the tumbling capsules. The temperature of the capsules should be approximately 28° C.

Using a peristaltic pump feeding at the rate of 18 ml/minute, the coating solution is sprayed on the capsules tumbling in the coating device. The solution is atomized using a Spraying Systems Company spray gun model VAU-316SS or similar device and 45 p.s.i. atomizing air. The temperature of the drying air should be approximately 39° C. while the capsules are being sprayed. All of the coating solution is sprayed on the capsules. The capsules are allowed to tumble slowly until thoroughly dry.

The following formulation is used as an optional protective sealing coat. In an appropriate container, 8.604 gm of hypromellose, 1.225 gm of Povidone and 1.050 gm of Vanilla Flavor is mixed with 79.167 gm of purified water. After mixing for about twenty minutes, 2.450 gm of Poltethylene Glycol and 0.642 gm of Polysorbate 80 is added to this mixture and remixed for an additional fifteen minutes. Using the spray equipment described above for the application of the enteric coating solution, the protective sealing layer is sprayed on the tumbling capsules.

While this invention has been described with reference to certain specific embodiments, it will be recognized by those skilled in the art that many variations are possible without departing from the scope and spirit of the invention, and it will be understood that it is intended to cover all changes and modifications of the invention, disclosure and for the purpose of illustration, which do not constitute departures from the spirit and scope of the invention.

1. A coated liquid-filled soft capsule comprising:
a) a liquid fill,
b) a soft capsule shell encapsulating said liquid fill, said capsule shell formed from a material which further comprises a colorant incorporated in said material in an amount sufficient to provide a visual contrast between said capsule shell and any liquid fill that escapes from said capsule shell and resides on an exterior surface of said capsule shell, and
c) a coating applied on said exterior surface.

2. The capsule of claim 1, wherein said material comprises gelatin.

3. The capsule of claim 2, wherein said gelatin is natural gelatin.

4. The capsule of claim 2, wherein said gelatin is chemically or enzymatically modified gelatin.

5. The capsule of claim 1, wherein said material comprises a heat sealable polymer.

6. The capsule of claim 1, wherein said material further comprises an extender.

7. The capsule of claim 1, wherein said extender is selected from the group consisting of natural and modified natural biopolymers and synthetic polymers.

8. The capsule of claim 7, wherein said natural biopolymer is, starch, starch derivatives, bacterial polysaccharides or gum.

9. The capsule of claim 7, wherein said modified natural biopolymer is modified cellulose.

10. The capsule of claim 1, wherein said capsule shell comprises at least about 20% material.

11. The capsule of claim 2, wherein said capsule shell comprises at least about 25% material.

12. The capsule of claim 2, wherein said capsule shell comprises at least about 30% material.

13. The capsule of claim 2, wherein said capsule shell comprises at most about 85% material.

14. The capsule of claim 2, wherein said capsule shell comprises at most about 70% material.

15. The capsule of claim 2, wherein said capsule shell comprises at most about 60% material.

16. The capsule of claim 1, wherein said liquid fill comprises an active pharmaceutical agent, a vitamin, a mineral, an antioxidant, an enzyme, an immunostimulant, a weight loss product, an energy product or a nutritional supplement.

17. The capsule of claim 16, wherein said liquid fill comprises a nutritional oil.

18. The capsule of claim 17, wherein said nutritional oil further comprises a stabilizer.

19. The capsule of claim 18, wherein said stabilizer is an antioxidant.

20. The capsule of claim 17, wherein said nutritional oil comprises omega-3 fatty acids.

21. The capsule of claim 17, wherein said nutritional coil comprises omega-3 fatty acids and omega-6 fatty acids.

22. The capsule of claim 17, wherein said nutritional oil comprises omega-3 fatty acids, omega-6 fatty acids, essential fatty acids, or combinations thereof.

23. The capsule of claim 17, wherein said nutritional oil comprises an essential fatty acid.

24. The capsule of claim 17, wherein said nutritional oil is fish oil and/or flaxseed oil.

25. The capsule of claim 1, wherein said liquid fill further comprises a colorant.

26. The capsule of claim 1, wherein said colorant is selected from the group consisting of
i) a pigment,
ii) a dye, and
iii) a combination of a pigment and a dye.

27. The capsule of claim 1, wherein said colorant is selected from the group consisting of titanium dioxide, zinc oxide, iron oxides, iron hydroxides, calcium carbonate, calcium sulfate, curcumin, riboflavin, tartrazine, quinoline yellow, carmoisine, indigo carmine, chlorophylls, copper complexes of chlorophylls, lissamine green, caramel, charcoal, carotenoids, xanthophylls, anthocyanins, alumina, aluminum powder, annatto extract, bismuth oxychloride, bronze powder, canthaxanthin, chromium-cobalt-aluminum oxide, chromium hydroxide green, cochineal extract, carmine, copper powder, ferric ammonium citrate, ferric ammonium ferrocyanide, ferric ferrocyanide, guanine, logwood extract, mica, potassium sodium copper chlorophyllin, pyrogallol, ptrophyllite, talc, annatto extract, FD&C dyes, aluminum lake forms of FD&C dyes, D&C dyes, and aluminum lake forms of D&C dyes.

28. The capsule of claim 27, wherein said colorant is titanium dioxide.

29. The capsule of claim 1, wherein said gelatin capsule has a wet seal thickness of at least about 0.006 inches.

30. The capsule of claim 1, wherein said gelatin capsule has a wet shell thickness of at least about 0.025 inches to about 0.035 inches.

31. The capsule of claim 1, further comprising an exterior finishing coat.

32. The capsule of claim 1, wherein the coating is an enteric coating.

33. The capsule of claim 1, wherein the coating comprises cellulose, vinyl, glycol, acrylic or carbohydrate polymers.

34. The capsule of claim 1, wherein the coating further comprises a plasticizer.

35. The capsule of claim 1, wherein the coating further comprises a processing aid.

36. The capsule of claim 1, wherein the coating further comprises an edible fragrant substance.

37. The capsule of claim 1, wherein the coating substantially lacks a colorant.

38. An enteric coated liquid-filled soft capsule comprising:
a) a liquid fill,
b) a soft capsule shell encapsulating said liquid fill, said capsule shell comprising gelatin and a colorant in said gelatin in an amount sufficient to provide a visual contrast between said capsule shell and any liquid fill that escapes from said capsule shell and resides on an exterior surface of said capsule shell, and
c) an enteric coating applied on said exterior surface.

39.-77. (canceled)
